(19) United States
(12) Patent Application Publication
(10) Pub. No.: US 2015/0265651 A1
(43) Pub. Date: Sep. 24, 2015

(54) Herbal Composition for the Prevention and Treatment of TNF-ALPHA Mediated Diseases

(71) Applicant: PIRAMAL ENTERPRISES LIMITED, Mumbai (IN)

(72) Inventors: Somesh Sharma, Los Altos, CA (US); Geetanjali Chandrashekhar Chimote, Mumbai (IN); Becky M. Thomas, Mumbai (IN); Jacqueline Vinodkumar Trivedi, Mumbai (IN); Jayasree Sreenivasan, Mumbai (IN); Mahesh G. Jadhav, Mumbai (IN)

(73) Assignee: PIRAMAL ENTERPRISES LIMITED, Mumbai (IN)

(21) Appl. No.: 14/435,669

(22) PCT Filed: Oct. 22, 2013
(86) PCT No.: PCT/IB2013/059538

§ 371 Date: Apr. 14, 2015

Related U.S. Application Data
(60) Provisional application No. 61/717,131, filed on Oct. 23, 2012.

(51) Int. Cl. A61K 33/30; A61K 31/09; A61K 36/9066; A61K 36/28

(52) U.S. Cl. CPC A61K 33/30; A61K 36/28; A61K 31/09; A61K 36/9066

(57) ABSTRACT

The present invention relates to a herbal composition for use in the prevention or treatment of a disease mediated by TNF-α. The present invention also provides a method for the preparation of the herbal composition and methods for using such herbal composition.

### FIELD OF THE INVENTION

The present invention relates to a herbal composition for use in the prevention or treatment of diseases mediated by TNF-α (Tumor necrosis factor-alpha). The present invention also provides a method for the preparation of the herbal composition and methods for using such a herbal composition.

### BACKGROUND OF THE INVENTION

Tumor Necrosis Factor-α (TNF-α) or cachexin or cachectin is a macrophage secreted polypeptide hormone. It is a primary mediator in the pathogenesis of infection, injury and inflammation. TNF-α converting enzyme (TACE, also known as ADAM-17) mediates release of TNF from the cell surface and is also involved in the processing of TNF receptors that can neutralize the actions of TNF. TACE may act either as pro-inflammatory or as anti-inflammatory, depending on whether it acts on an effector (e.g. macrophage) or target (e.g. endothelial) cell (J. Pathol., 2008, 214, 149-160).

TNF is a major inflammatory cytokine and has central role in host defense and inflammation (Nature Reviews Immunology, 2003, 3, 745-756). To exert its biological function, TNF interacts with two different receptors, designated as TNFR1 and TNFR2, which are differently expressed in cells and tissues and initiate both distinct and overlapping signal transduction pathways. These diverse signaling cascades lead to a range of cellular responses, which include cell death, survival, differentiation, proliferation and migration. Based on the cell culture work and studies with receptor knock-out mice, it has been found that both the pro-inflammatory and the programmed cell death pathways that are activated by TNF, and associated with tissue injury, are largely mediated through TNFR1 (J. Pathol., 2008, 214, 149-160).

TNFR1 is also known to activate other signaling responses, these include, ras-raf-MEK1-ERK-1,2 pathway and phosphatidylinositol-3-kinase (PI3K), which in turn activates Akt. ERK-1, 2 and Akt are generally associated with cell survival and proliferation. TNF-α has a role in the recruitment of inflammatory cells in animal models of glomerular injury. It has been reported that administration of TNF-α stimulates cyst formation in adult PKD2^{+/-} heterozygous mice (Curr Opin Nephrol Hypertens., 2009, 18(2), 99-106). TNF-α also stimulates cyst formation, in vivo, in PKD2^{+/-} mice (Nature Medicine, 2008, 14(8), 863-868).

TNF-α has been implicated as a mediator in numerous disease conditions including inflammatory bowel disease, rheumatoid arthritis, juvenile rheumatoid arthritis, psoriatic arthritis, osteoarthritis, refractory rheumatoid arthritis, chronic non-rheumatoid arthritis, osteoporosis/bone resorption, Crohn's disease, ulcerative colitis, psoriasis, Behcet's disease, ankylosing spondylitis, systemic lupus erythematosus, allergic asthma and renal cystic diseases such as polycystic kidney disease (PKD).

PKD is the most common genetic cause of chronic kidney disease and a leading cause for end-stage renal failure. It is a common indication for dialysis or renal transplantation. PKD is characterized by the accumulation of numerous fluid filled cysts in the renal parenchyma, which progressively increases in size. The enlarging cysts compress surrounding normal nephrons resulting in a decline of renal function. PKD is inherited either as an autosomal dominant trait (ADPKD) or autosomal recessive trait (ARPKD). ADPKD has an incidence of 1:400-1:1000 and is one of the most common monogenic disorders. Characteristic symptoms of ADPKD include proteinurea, abdominal pain and palpable kidneys followed by haematuria, hypertension, pyurea, uremia and calculi.

ARPKD primarily affects infants and children and has been estimated to have an incidence of 1:20,000. The most extreme cases are associated with the potter's phenotype, consisting of pulmonary hypoplasia, characteristic facies and spine and limb abnormalities. A significant proportion of cases (up to 30%) die by the neonatal period, primarily of respiratory insufficiency. In the survivors, hypertension and renal insufficiency, including end-stage renal disease (ESRD) (up to a third of children require renal replacement therapy), are the major signs of renal disease (Annu Rev Med. 2009, 60, 321-337).

TNF-α has also been implicated as a mediator in neurological disorders such as Alzheimer's disease (AD), Parkinson's disease, stroke and head trauma (J Neuroinflammation, 2012, 9, 106). The levels of TNF-α have been found to be elevated within the CSF of AD patients as much as 25 folds (J. Clin Immunol., 1999, 19(4), 223-30).

Certain monoclonal antibodies (e.g.; infliximab, adalimumab and etanercept) are currently used in the treatment of diseases mediated by TNF-α such as Crohn's disease and rheumatoid arthritis. However, there are limited options for the clinical management of PKD. Management of patients with PKD consists of supportive measures, such as analgesics for pain; antibiotics for cyst infection; blood pressure control, in pre-ESRD patients and dialysis or transplantation in patients with ESRD. Certain investigational drugs are currently being studied in preclinical and clinical trials, such as tolvaptan, rapamycin, roscovitine, triptolide, and octreotide.

Further, the monoclonal antibodies are associated with certain adverse effects. Since these drugs are administered only by parenteral route, they are associated with adverse effects such as immunogenicity, acute and delayed infusion and injection-site reactions. These drugs are also associated with autoimmune disorders and infection. Tuberculosis and the risk of reactivating latent tuberculosis associated with these drugs are of particular concern. Rare occurrences of demyelination and hematologic abnormalities were also seen in patients receiving adalimumab for rheumatoid arthritis. Moreover these drugs are expensive.

Thus, there is a need for improved and alternative medicaments for the prevention or treatment of diseases caused by increased TNF-α activity.

The inventors of the present invention provide herewith a herbal composition comprising at least two active ingredients selected from an extract of flowering and fruiting heads of Sphaeranthus indicus, Yashada bhasma and curcumin or curcumin containing substance for the prevention or treatment of diseases caused by increased TNF-α activity.

Sphaeranthus indicus is a plant species belonging to the genus Sphaeranthus and family Asteraceae. It is an aromatic herb with purple or pink flowers. It is a nervine, diuretic, vermicide and a blood purifier. It is used in various diseases like skin affections, filariasis, epilepsy, anemia, obesity and diseases of the anus and vagina (International Journal of Drug Formulation & Research, 2010, 1 (iii), 113-133).

Yashada bhasma is a traditional herbomineral preparation mainly comprising of zinc. In some Ayurvedic texts, Yashada bhasma has been recommended in case of tuberculosis, common cold, indigestion, weakness, diabetes and anemia (Ancient Science of Life, 1996, XVI (2), 118-121 and Pharmaceutico-analytical Study of Hatakakhya Rasa, Dissertation submitted to Rajiv Gandhi University of Health Sciences, Karnataka, Bangalore, 2009).

Curcumin is the major constituent of the Indian spice, turmeric. The curcumin in turmeric has been shown to stimulate the production of bile by the gallbladder. Curcumin is also a powerful antioxidant. Turmeric (Curcuma longa) is grown wild in the forests of Southern Asia including India, and has been known to treat a variety of ailments. It is used in both Ayurvedic and Chinese medicine as an anti-inflammatory, to treat digestive and liver problems, skin diseases, and wounds and is a well-known antiseptic. It also helps prevent flatulence. Turmeric, being rich in iron is useful in anemia (Archives of Applied Science Research, 2009, 1 (2) 86-108).

### SUMMARY OF THE INVENTION

According to one aspect of the present invention, there is provided a herbal composition comprising at least two active ingredients selected from an extract of flowering and/or fruiting heads of Sphaeranthus indicus, Yashada bhasma and curcumin or a curcumin containing substance.

According to another aspect, the herbal composition of the present invention is provided for use in the prevention or treatment of a disease mediated by TNF-α.

According to another aspect, there is provided a method for the prevention or treatment of a disease mediated by TNF-α comprising administering to a subject in need thereof a therapeutically effective amount of the herbal composition of the present invention.

According to another aspect, there is provided a method for the prevention or treatment of external infection selected from bacterial infection, yeast infection, fungal infection and viral infection comprising administering to a subject in need thereof a therapeutically effective amount of the herbal composition of the present invention.

According to another aspect, there is provided a method for the preparation of the herbal composition of the present invention.

According to another aspect of the present invention, there is provided a method for the manufacture of a medicament, comprising the herbal composition for use in the treatment of a disease mediated by TNF-α.

### BRIEF DESCRIPTION OF THE DRAWINGS

FIG. 1 is a graphical representation showing effect of different doses of Yashada bhasma on TNF-α inhibition.

FIG. 2A is a graphical representation showing the effect of different doses of turmeric (containing 25% curcumin) on TNF-α inhibition.

FIG. 2B is a graphical representation showing the effect of different doses of turmeric on TNF-α inhibition.

FIG. 3 is a graphical representation showing the effect of different doses of Sphaeranthus indicus and Yashada bhasma alone and in combination on TNF-α inhibition.

FIG. 4A is a graphical representation showing the effect of the herbal composition comprising Sphaeranthus indicus, Yashada bhasma and turmeric (containing 25% curcumin) on TNF-α inhibition.

FIG. 4B is a graphical representation showing the effect of the herbal composition comprising Sphaeranthus indicus, Yashada bhasma and turmeric on TNF-α inhibition.

FIG. 5Aa is a graphical representation showing the effect of the herbal composition comprising Sphaeranthus indicus and curcumin on renal cyst size.

FIG. 5Ab is a graphical representation showing the effect of the herbal composition comprising Sphaeranthus indicus and turmeric on renal cyst size.

FIG. 5Ac, FIG. 5Ba and FIG. 5Ca is a graphical representation showing the effect of the herbal composition comprising Sphaeranthus indicus, curcumin and Yashada bhasma on renal cyst size.

FIG. 5Ad, FIG. 5Bb and FIG. 5Cb is a graphical representation showing the effect of the herbal composition comprising Sphaeranthus indicus, turmeric and Yashada bhasma on renal cyst size.

FIG. 6 is a graphical representation showing the effect of the herbal composition comprising Sphaeranthus indicus, turmeric and Yashada bhasma on chronic ear inflammation of mice.

### DETAILED DESCRIPTION OF THE INVENTION

The general terms used hereinbefore and hereinafter preferably have within the context of this disclosure the following meanings, unless otherwise indicated. Thus, the definitions of the general terms as used in the context of the present invention are provided herein below:

The singular forms “a,” “an,” and “the” include plural reference unless the context clearly dictates otherwise.

The term “therapeutically effective amount”, as used herein refers to the amount of the herbal composition of the present invention, when administered to a subject in need thereof, is sufficient to inhibit the activity of TNF-α such that the disease mediated by TNF-α is reduced, treated or alleviated.

The term “subject” as used herein, refers to an animal, preferably a mammal, most preferably a human, who is in the need of treatment of a disease mediated by TNF-α. The term subject may be interchangeably used with the term patient in the context of the present invention.

The term “mammal” as used herein is intended to encompass humans, as well as non-human mammals. Non-human mammals include but are not limited to domestic animals, such as cows, pigs, horses, dogs, cats, rabbits, rats and mice, and non-domestic animals.

The term “treat” or “treatment” as used herein includes prophylaxis, amelioration [i.e., reduction in the severity of the disease or accompanying symptoms], regression or curing of a disease mediated by TNF-α.

The term “prevention” as used herein refers to prophylactic administration of the herbal composition of the present invention to a subject in need thereof. The term also refers to complete or partial prevention or attenuation of the disease or one or more symptoms of the disease mediated by TNF-α.

The term “disease having an inflammatory component” as used herein refers to a disease, wherein inflammation is a common or a significant symptom. Diseases with inflammatory component include, but are not limited to, neurodegenerative diseases such as Alzheimer's disease, vascular diseases such as atherosclerosis, metabolic disorders such as type-2 diabetes and kidney diseases such as nephritis.

The term “a disease mediated by TNF-α” as used herein refers to a condition in which TNF-α has a significant role in the onset or progression of that condition. Examples of conditions mediated by TNF-α include, but are not limited to, coronary heart disease, atherosclerosis, hyperlipidemia, ischemia-reperfusion injury, stroke, cachexia, hypertension, vasculitis, Wegener's granulomatosis, multiple sclerosis, common variable immunodeficiency (LVID), skin delayed type hypersensitivity disorders, ankylosing spondylitis, rheumatoid arthritis, juvenile rheumatoid arthritis, psoriatic arthritis, osteoarthritis, refractory rheumatoid arthritis, chronic non-rheumatoid arthritis, chronic graft-versus-host disease, psoriasis, conjunctivitis, Crohn's disease, ulcerative colitis, Behcet's disease, inflammatory bowel disease, osteoporosis/bone resorption, chronic obstructive pulmonary disease or asthma.

As used herein the term “pharmaceutically acceptable” is meant that the carrier, diluent, excipients, and/or salt must be compatible with the other ingredients of the formulation, and not deleterious to the recipient thereof. As used herein the term “pharmaceutically acceptable” also means that the compositions or dosage forms are within the scope of sound medical judgment, suitable for use for an animal or human without excessive toxicity, irritation, allergic response, or other problem or complication, commensurate with a reasonable benefit/risk ratio.

According to one embodiment of the present invention, there is provided a herbal composition for the prevention or treatment of a disease mediated by TNF-α.

According to another embodiment, the present invention relates to a herbal composition comprising at least two active ingredients selected from an extract of flowering and fruiting heads of Sphaeranthus indicus, Yashada bhasma and curcumin or curcumin containing substance.

According to yet another embodiment, the present invention relates to a herbal composition comprising active ingredients selected from an extract of flowering and fruiting heads of Sphaeranthus indicus and Yashada bhasma.

According to yet another embodiment, the present invention relates to a herbal composition comprising active ingredients selected from an extract of flowering and fruiting heads of Sphaeranthus indicus and curcumin or curcumin containing substance.

According to another embodiment, the present invention relates to the herbal composition comprising active ingredients selected from Yashada bhasma and curcumin or curcumin containing substance.

According, to yet another embodiment, the herbal composition of the present invention recited in one or more embodiments stated above may further contain a pharmaceutically acceptable carrier or excipient.

According to another aspect of the present invention, there is provided a herbal composition comprising an extract of flowering and fruiting heads of Sphaeranthus indicus, Yashada bhasma and curcumin containing substance in a ratio ranging from 1:1:1 to 16:8:5.

According to another aspect of the present invention, there is provided a herbal composition, wherein the composition comprises active ingredients selected from an extract of flowering and fruiting heads of Sphaeranthus indicus and curcumin containing substance in the ratio ranging from 1:1 to 10:1.

According to another aspect of the present invention, there is provided a herbal composition, wherein the composition comprises active ingredients selected from an extract of flowering and fruiting heads of Sphaeranthus indicus, Yashada bhasma and curcumin in the ratio ranging from 1:1:1 to 15:1:1.

According to another aspect of the present invention, there is provided a herbal composition, wherein the composition comprises active ingredients selected from an extract of flowering and fruiting heads of Sphaeranthus indicus and Yashada bhasma in the ratio ranging from 1:1 to 1:5.

According to another aspect of the present invention, there is provided a herbal composition, wherein the composition comprises active ingredients selected from an extract of flowering and fruiting heads of Sphaeranthus indicus and curcumin in the ratio ranging from 1:1 to 10:1.

According to another embodiment of the present invention, the curcumin containing substance comprises of about 20% to about 30% of curcumin.

According to yet another embodiment of the present invention, the curcumin containing substance is turmeric.

According to yet another embodiment of the present invention, the active ingredients of the herbal composition provided herein exhibit synergistic effect in the treatment of inflammatory disease or a disease having an inflammatory component or a renal cystic disease.

According to another aspect of the present invention, there is provided a method for the prevention or treatment of a disease mediated by TNF-α, comprising administering to a subject in need thereof a therapeutically effective amount of the herbal composition.

According to another embodiment of the present invention, there is provided a method for the prevention or treatment of a disease mediated by TNF-α, comprising administering to a subject in need thereof a therapeutically effective amount of the herbal composition comprising at least two active ingredients selected from an extract of flowering and fruiting heads of Sphaeranthus indicus, Yashada bhasma and curcumin or curcumin containing substance.

According to another aspect of the present invention, there is provided a herbal composition for use in the prevention or treatment of a disease mediated by TNF-α.

According to another embodiment of the present invention, there is provided a herbal composition comprising at least two active ingredients selected from an extract of flowering and fruiting heads of Sphaeranthus indicus, Yashada bhasma and curcumin or curcumin containing substance; for use in the prevention or treatment of a disease mediated by TNF-α, comprising administering to a subject in need thereof a therapeutically effective amount of the herbal composition.

According to another aspect of the present invention, there is provided a method for the manufacture of a medicament comprising the herbal combination, for use in the prevention or treatment of a disease mediated by TNF-α.

According to another embodiment of the present invention, there is provided a method for the manufacture of a medicament for prevention or treatment of a disease mediated by TNF-α, comprising administering to a subject in need thereof a therapeutically effective amount of the herbal composition comprising at least two active ingredients selected from an extract of flowering and fruiting heads of Sphaeranthus indicus, Yashada bhasma and curcumin or curcumin containing substance.

According to another embodiment of the present invention, the disease mediated by TNF-α is an inflammatory disease or a disease having an inflammatory component, or a renal cystic disease.

According to another embodiment of the present invention, there is provided a method for the prevention or treatment of an inflammatory disease or a disease having an inflammatory component, selected from coronary heart disease, atherosclerosis, hyperlipidemia, ischemia-reperfusion injury, stroke, cachexia, hypertension, vasculitis, Wegener's granulomatosis, liver cirrhosis, liver fibrosis, hepatitis, diabetes, juvenile diabetes, multiple sclerosis, common variable immunodeficiency (LVID), Alzheimer's disease, epilepsy, skin delayed type hypersensitivity disorders, ankylosing spondylitis, rheumatoid arthritis, juvenile rheumatoid arthritis, psoriatic arthritis, osteoarthritis, refractory rheumatoid arthritis, chronic non-rheumatoid arthritis, chronic graft-versus-host disease, psoriasis, conjunctivitis, Crohn's disease, ulcerative colitis, Behcet's disease, inflammatory bowel disease, nephritis, osteoporosis/bone resorption, chronic obstructive pulmonary disease, bronchitis or asthma.

According to another embodiment of the present invention, there is provided a method for the prevention or treatment of an inflammatory disease or a disease having an inflammatory component, selected from rheumatoid arthritis, juvenile rheumatoid arthritis, psoriatic arthritis, osteoarthritis, refractory rheumatoid arthritis, chronic non-rheumatoid arthritis, psoriasis, inflammatory bowel disease, Crohn's disease, ulcerative colitis, osteoporosis/bone resorption, atherosclerosis or ankylosing spondylitis.

According to another embodiment of the present invention, there is provided a method for the prevention or treatment of renal cystic disease, comprising administering to a subject in need thereof a therapeutically effective amount of the herbal composition.

According to yet another embodiment of the present invention, the renal cystic disease is polycystic kidney disease (PKD).

According to yet another embodiment of the present invention, the polycystic kidney disease is autosomal dominant polycystic kidney disease (ADPKD) or autosomal recessive polycystic kidney disease (ARPKD).

According to yet another embodiment of the present invention, there is provided a method for the prevention or treatment of the diseases selected from Von Hippel Lindau disease, nephronophthisis (NPHP), Bardet-Biedl syndrome (BBS), Joubert syndrome (JBTS) and Meckel-Gruber syndrome (MKS) comprising administering to a subject in need thereof a therapeutically effective amount of the herbal composition of the present invention.

According to further embodiment of the present invention, there is provided a method for the prevention or treatment of an infection selected from bacterial infection, yeast infection, fungal infection and viral infection, comprising administering to a subject in need thereof a therapeutically effective amount of the herbal composition of the present invention. Bacterial infection includes infection by gram positive, gram negative, anaerobic or aerobic bacteria. Examples of an infection include, but not limited to, skin infections and nail fungus.

According to further embodiment of the present invention, there is provided a method for using the herbal composition as a food supplement.

According to further embodiment of the present invention, there is provided a method for using the herbal composition as a food supplement for the prevention, treatment or decrease in the prevalence of anemia.

According to further embodiment of the present invention, there is provided a method for using the herbal composition as a food supplement for the prevention or treatment of dyspepsia and indigestion.

There is provided a herbal composition which comprises a therapeutically effective amount of at least two active ingredients selected from an extract of flowering and fruiting heads of Sphaeranthus indicus, Yashada bhasma and curcumin or a curcumin containing substance in association with a pharmaceutically acceptable carrier. The composition can further include pharmaceutically active compound, which has inhibitory effect on TNF-α. The pharmaceutically active compound can be selected from, but not limited to, tolvaptan, rapamycin, roscovitine, triptolide, octreotide and etanercept.

The herbal composition may contain about 10% to 100%, for example about 40% to 90%, or from about 60% to about 80% by weight of the herbal composition. The amount of the herbal composition in the pharmaceutical dosage forms may be from about 50 mg to about 1000 mg.

The herbal composition may contain 10% to 80%, or from about 20% to 70%, or from about 40% to about 60% by weight of extract of Sphaeranthus indicus.

The herbal composition may contain 10% to 60%, or from about 20% to 50%, or from about 30% to about 40% by weight of extract of Yashada Bhasma.

The herbal composition may contain 1% to 40%, or from about 5% to 20%, or from about 5% to about 15% by weight of extract of turmeric.

According to another embodiment of the present invention, there is provided a herbal composition, comprising (a) from about 30% to 70% by weight of an extract of flowering and fruiting heads of Sphaeranthus indicus; (b) from about 20% to about 40% of Yashada bhasma; and (c) from about 3% to about 40% of curcumin or curcumin containing substance.

According to further embodiment of the present invention, there is provided a herbal composition, comprising (a) from about 10% to 50% by weight of an extract of flowering and fruiting heads of Sphaeranthus indicus; and (b) from about 50% to about 85% of Yashada bhasma.

The herbal composition may be administered orally, parenterally (including intravenous, subcutaneous, intramuscular, intravascular or infusion), sublingually, via buccal route, transdermally, rectally, transmucosally, topically or via inhalation. The herbal composition may be administered by any route appropriate to the condition to be treated. It will be appreciated that the preferred route may vary with the condition of the subject being treated. For example, for skin infections, the herbal composition may be administered orally or topically.

The herbal compositions described herein may be in a form suitable for oral administration, for example as a tablet or capsule; for parenteral injection (including intravenous, subcutaneous, intramuscular, intravascular or infusion) for example as a sterile solution, suspension or emulsion in oily or aqueous vehicles; for topical administration for example as an ointment, cream, gel, lotions or collodion; for rectal administration for example as a suppository or a pessary.

For oral use, the herbal composition may be administered, for example, in the form of tablets, capsules, lozenges, powders, powder form for reconstitution, dispersible granules, cachets, aqueous or oily suspensions, aqueous solutions, emulsions, syrups or elixirs. Orally administered compositions may contain one or more optional agents, for example, sweetening agents such as fructose, aspartame or saccharin; flavoring agents such as peppermint, oil of wintergreen, or cherry; coloring agents; and preserving agents, to provide a pharmaceutically palatable preparation. Selectively permeable membranes surrounding an osmotically active driving compound are also suitable for oral administration of the herbal composition of present invention. Oral compositions can include standard vehicles such as mannitol, lactose, starch, magnesium stearate, talc, sodium saccharine, sugar, cellulose, magnesium carbonate, etc. Such vehicles are preferably of pharmaceutical grade.

For intramuscular, intraperitoneal, subcutaneous and intravenous use, sterile vehicles are usually employed, and the pH of the solutions should be suitably adjusted and buffered. The parenteral administration may be by injection or continuous infusion.

For ointments and creams, the herbal composition may be formulated in oil-in-water or water-in-oil base.

For rectal use, the herbal composition may be administered in the form of suppositories or pessaries. A suppository comprises of at least two active ingredients selected from an extract of flowering and fruiting heads of Sphaeranthus indicus, Yashada bhasma and curcumin or curcumin containing substance, a suitable suppository base and additives such as preservatives, antioxidants, emulsifiers and the like. Suitable suppository bases include natural or synthetic triglycerides or paraffin hydrocarbons.

Suppositories are solid bodies for insertion into the rectum which melt or soften at body temperature releasing one or more pharmacologically or therapeutically active ingredients. Pharmaceutically acceptable substances utilized in rectal suppositories are bases or vehicles and agents to raise the melting point. Examples of bases include cocoa butter (theobroma oil), glycerin-gelatin, carbowax (polyoxyethylene glycol) and appropriate mixtures of mono-, di- and triglycerides of fatty acids. Combinations of the various bases may be used. Agents to raise the melting point of suppositories include spermaceti and wax. Suppositories may be prepared either by the compressed method or by molding.

The herbal composition may be formulated as a nanoparticle solution or a suspension.

Therapeutically effective dose of the herbal composition described herein depends at least on the nature of the condition being treated, the mode of delivery, the pharmaceutical formulation, metabolism, other factors such as sex, weight, age and condition of the patient and eventually will be determined by the clinician using conventional dose escalation studies. It can be expected to be from about 1 mg/kg to about 100 mg/kg body weight per day; particularly, from about 1 mg/kg to about 50 mg/kg body weight per day. The desired dose may be administered as a single dose or as multiple doses.

According to another aspect of the present invention, there is provided a method for the preparation of the herbal composition of the present invention.

According to another embodiment of the present invention, the herbal composition of the present invention is prepared by dissolving at least two active ingredients selected from an extract of flowering and fruiting heads of Sphaeranthus indicus, Yashada bhasma and curcumin or curcumin containing substance, in a suitable solvent selected from dimethyl sulfoxide, ethanol or water and in presence of an emulsifying agent selected from Tween-80.

The extracts of the active ingredients may be produced by conventional extraction techniques such as solvent extraction using extraction solvents, including but not limited to, alcohol such as ethanol, water, mixture of ethanol and water; percolation by water; maceration by water or alcoholic solvents and non-conventional techniques such as soxhlet extraction; lyophilization; sonication and microwave assisted extraction.

### Experimental

Unless otherwise stated all temperatures are in degree Celsius. Also, in these examples and elsewhere, abbreviations have the following meanings:

List of abbreviations

| | |
|---|---|
| ° C. | Degree centigrade |
| DMSO | Dimethyl sulfoxide |
| ELISA | Enzyme Linked ImmunoSorbent Assay |
| g | Gram(s) |
| L | Litre(s) |
| KCl | Potassium Chloride |
| LPS | Lipopolysaccharide |
| mg | milligram |
| mL | millilitre |
| μl | Microlitre |
| μM | Micromolar |
| mM | Millimolar |
| i.p | Intraperitoneally |
| p.o | Per orally |
| rpm | Rotations per minute |
| SEM | Standard Error of Mean |
| w/w | Weight/weight |

### Constituents of the Herbal Composition

Sphaeranthus indicus:

Dried flowering and fruiting heads of Sphaeranthus indicus (200 g) were pulverized. The powdered material was extracted using methanol (2.5 L) by stirring at 60° C. for 3 hours. The extract was filtered under vacuum. This extraction process was repeated two more times. The extracts were combined and concentrated.
Yield: 23.29 g (11.65% w/w).

### Yashada Bhasma:

Yashada bhasma (Calx of Yashada i.e. zinc) was obtained from Shree Dhootapapeshwar Ltd, Panvel, India.

### Turmeric:

Turmeric (Curcuma longa) obtained from commercial sources was used as such. Turmeric (containing 25% curcumin) was obtained from Sanat Products Limited, Sikandrabad, Uttar Pradesh, India.

### Curcumin:

Curcumin was obtained from Sigma Aldrich, USA.

The following examples are illustrative, but not limiting the scope of the present invention. Reasonable variations, such as those occur to reasonable artisan, can be made herein without departing from the scope of the present invention. Also in describing the invention, specific terminology is employed for the sake of clarity. However, the invention is not intended to be limited to the specific terminology so selected. It is to be understood that each specific element includes all technical equivalents which operate in a similar manner to accomplish a similar purpose.

### In Vivo Experiments:

All experiments were carried out in accordance with the guidelines of Committee for the Purpose of Control and Supervision of Experiments on Animals (CPCSEA) and with the approval of Institutional Animal Ethics Committee (IAEC) in Piramal Enterprises Limited, Goregaon, Mumbai, India.

In vivo efficacy of the herbal composition of the present invention on proinflammatory cytokine TNF-α was studied on Balb/c mice.

Animals Used for the following Experiments:

Balb/c male mice, 8 to 10 weeks old, weighing 18 to 22 g (Animal House facility of Piramal Enterprises Limited, Mumbai). Animals were housed in individually ventilated cage under specified pathogen-free conditions maintained at 22 to 25° C. and 55 to 70% humidity, with a 12-hour light/12-hour dark cycle. The mice were acclimatized for a period of minimum two days before experimentation. Animals were handled in a laminar flow hood. AH food and water was autoclaved. Mice had access to pelleted rodent diet (Pranav Agro industries, India) and UV-filtered water ad libitum.

### EXAMPLE 1

Effect of Yashada Bhasma (YB) on LPS induced TNF-α in Balb/c Mice

Dose preparation: Required amount of Yashada Bhasma (to achieve a concentration of 100 mg/mL, 75 mg/mL, 50 mg/mL, 25 mg/mL, 10 mg/mL, 5 mg/mL and 2.5 mg/mL) was triturated with Tween 80 until a smooth paste was formed. Carboxymethyl cellulose (0.5%) solution was added gradually with trituration to form a uniform suspension.

### Conditions for Storage of the Sample

Yashada Bhasma was stored at 22° C. to 25° C.

### Dosing

The mice were randomized (n=6) in the following ten groups of treatment:
i) Group 1: Control group: Mice were administered with vehicle (10 mL/kg, p.o.)
ii) Group 2: Mice administered once daily with 1 mg/kg of lipopolysaccharide i.p.
iii) Group 3: Mice administered once daily with 1.5 mg/kg of Rolipram p.o.
iv) Group 4: Mice administered once daily with 1000 mg/kg of Yashada Bhasma p.o.
v) Group 5: Mice administered once daily with 750 mg/kg of Yashada Bhasma p.o.
vi) Group 6: Mice administered once daily with 500 mg/kg of Yashada Bhasma p.o.
vii) Group 7: Mice administered once daily with 250 mg/kg of Yashada Bhasma p.o.
viii) Group 8: Mice administered once daily with 100 mg/kg of Yashada Bhasma p.o.
ix) Group 9: Mice administered once daily with 50 mg/kg of Yashada Bhasma p.o.
x) Group 10: Mice administered once daily with 25 mg/kg of Yashada Bhasma p.o.

### Treatment

Yashada bhasma (10 mL/kg), suspended in CMC-Tween 80 (Sigma Aldrich, US) was administered to all Balb/c mice of Groups 4 to 10. The positive control group of animals received Rolipram at 1.5 mg/kg. An hour later, lipopolysaccharide (LPS) dissolved in sterile pyrogen-free saline was administered intraperitoneally at a dose of 1 mg/kg to mice of all ten groups. After 1.5 hours, post LPS administration, animals were anaesthetized and blood samples were collected by retro orbital method into heparin containing (5 μl) eppendorf tubes. Plasma was separated by centrifugation at 4000 rpm and 4° C. and stored at −80° C. until analysis.

TNF-α levels in the blood samples were assayed using mouse TNF-α ELISA kits (BD Opt EIA) and the percent inhibition of TNF-α release in comparison to the control group was calculated.

TNF-α inhibition results are presented in FIG. 1.

Conclusion: Oral administration of Yashada bhasma demonstrated a dose dependent response in LPS-induced TNF-α production in Balb/c mice.

### EXAMPLE 2A

### Effect of Turmeric on LPS Induced TNF-α in Balb/c Mice

Dose preparation: Required amount of turmeric (containing 25% curcumin) (to achieve a concentration of 100 mg/mL, 50 mg/mL, 25 mg/mL, 10 mg/mL, 5 mg/mL, 2.5 mg/mL, 1.25 mg/mL, 0.625 mg/mL and 0.3125 mg/mL) was triturated with Tween 80 until a smooth paste was formed. Carboxymethyl cellulose (0.5%) solution was added gradually with trituration to form a uniform suspension.

### Conditions for Storage of the Sample

The sample of turmeric was stored at 22° C. to 25° C.

### Dosing

The mice were randomized (n=6) in the following twelve groups of treatment:
i) Group 1: Control group: Mice were administered with vehicle (10 mL/kg, p.o.)
ii) Group 2: Mice administered once daily with 1 mg/kg of lipopolysaccharide i.p.
iii) Group 3: Mice administered once daily with 1.5 mg/kg of Rolipram p.o.
iv) Group 4: Mice administered once daily with 1000 mg/kg of turmeric p.o.
v) Group 5: Mice administered once daily with 500 mg/kg of turmeric p.o.
vi) Group 6: Mice administered once daily with 250 mg/kg of turmeric p.o.
vii) Group 7: Mice administered once daily with 100 mg/kg of turmeric p.o.
viii) Group 8: Mice administered once daily with 50 mg/kg of turmeric p.o.
ix) Group 9: Mice administered once daily with 25 mg/kg of turmeric p.o.
x) Group 10: Mice administered once daily with 12.5 mg/kg of turmeric p.o.
xi) Group 11: Mice administered once daily with 6.25 mg/kg of turmeric p.o.
xii) Group 12: Mice administered once daily with 3.125 mg/kg of turmeric p.o.

### Treatment

Turmeric (10 mL/kg) was administered to all Balb/c mice of Groups 4 to 12. The positive control group of animals received Rolipram at 1.5 mg/kg. An hour later, lipopolysaccharide (LPS) dissolved in sterile pyrogen-free saline was administered intraperitoneally at a dose of 1 mg/kg to mice of all twelve groups. After 1.5 h, post LPS administration, animals were anaesthetized and blood samples were collected by retro orbital method into heparin containing (5 pp eppendorf tubes. Plasma was separated by centrifugation at 4000 rpm and 4° C. and stored at −80° C. until analysis.

TNF-α levels in the blood samples were assayed using mouse TNF-α ELISA kits (BD Opt EIA) and the percent inhibition of TNF-α release in comparison to the control group was calculated.

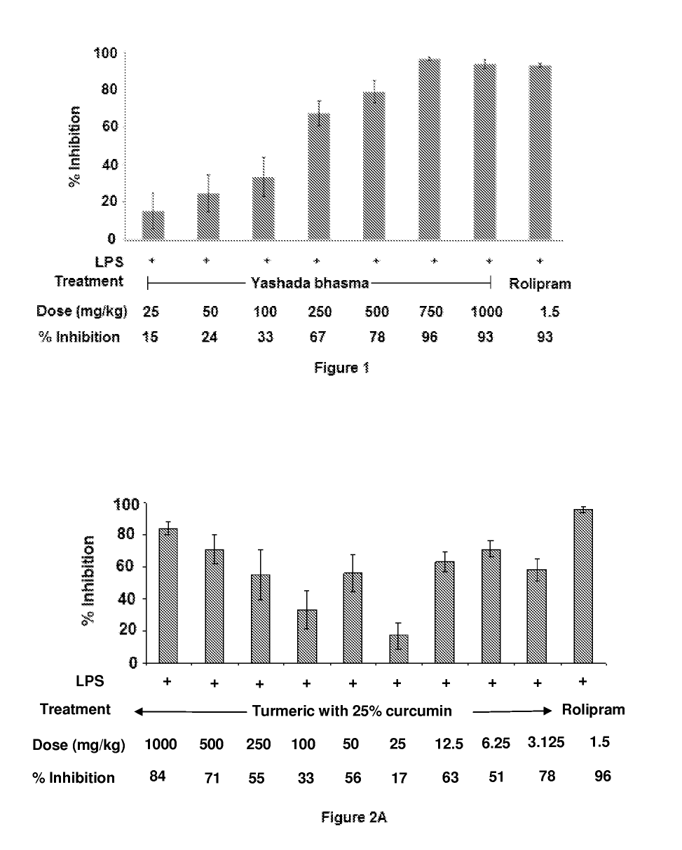

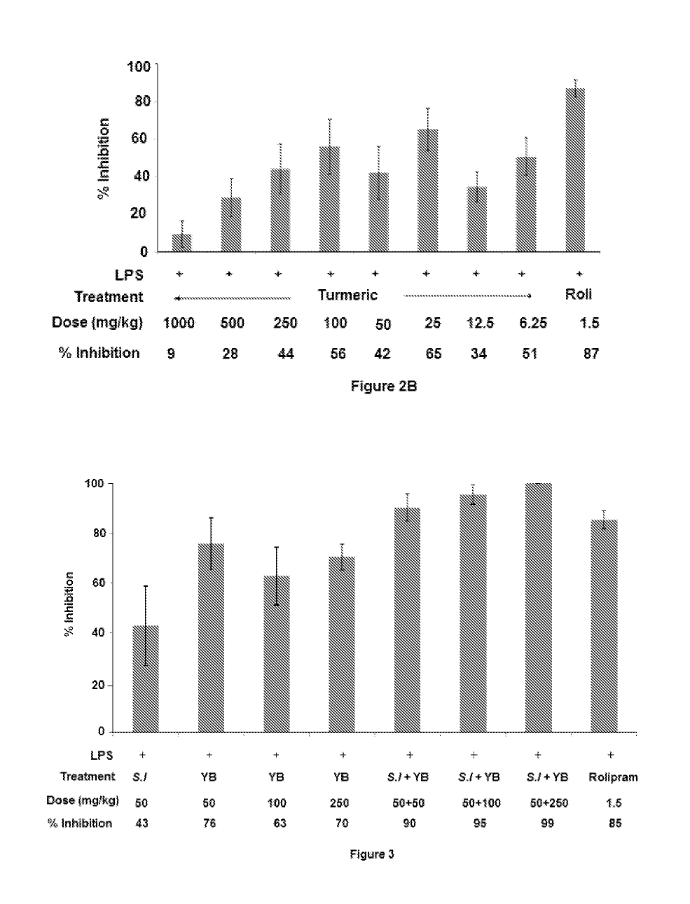

TNF-α inhibition results are presented in FIG. 2A.

Conclusion: Oral administration of turmeric with 25% curcumin at lower doses inhibited LPS induced TNF-α production in Balb/c mice.

### EXAMPLE 2B

### Effect of Turmeric on LPS Induced TNF-α in Balb/c Mice

Dose preparation: Required amount of turmeric (to achieve a concentration of 100 mg/mL, 50 mg/mL, 25 mg/mL, 10 mg/mL, 5 mg/mL, 2.5 mg/mL, 1.25 mg/mL and 0.625 mg/mL) was triturated with Tween 80 until a smooth paste was formed. Carboxymethyl cellulose (0.5%) solution was added gradually with trituration to form a uniform suspension.

### Conditions for Storage of the Sample

The sample of turmeric was stored at 22° C. to 25° C.

### Dosing

The mice were randomized (n=6) in the following eleven groups of treatment:
i) Group 1: Control group: Mice were administered with vehicle (10 mL/kg, p.o.)
ii) Group 2: Mice administered once daily with 1 mg/kg of LPS i.p.
iii) Group 3: Mice administered once daily with 1.5 mg/kg of Rolipram p.o.
iv) Group 4: Mice administered once daily with 1000 mg/kg of turmeric p.o.
v) Group 5: Mice administered once daily with 500 mg/kg of turmeric p.o.
vi) Group 6: Mice administered once daily with 250 mg/kg of turmeric p.o.
vii) Group 7: Mice administered once daily with 100 mg/kg of turmeric p.o.
viii) Group 8: Mice administered once daily with 50 mg/kg of turmeric p.o.
ix) Group 9: Mice administered once daily with 25 mg/kg of turmeric p.o.
x) Group 10: Mice administered once daily with 12.5 mg/kg of turmeric p.o.
xi) Group 11: Mice administered once daily with 6.25 mg/kg of turmeric p.o.

### Treatment

Turmeric (10 mL/kg) was administered to all Balb/c mice of Groups 4 to 11. The positive control group of animals received Rolipram at 1.5 mg/kg. An hour later, lipopolysaccharide (LPS) dissolved in sterile pyrogen-free saline was administered intraperitoneally at a dose of 1 mg/kg to the mice of all eleven groups. After 1.5 h, post LPS administration, animals were anaesthetized and blood samples were collected by retro orbital method into heparin containing (5 μl) eppendorf tubes. Plasma was separated by centrifugation at 4000 rpm and 4° C. and stored at −80° C. until analysis.

TNF-α levels in the blood samples were assayed using mouse TNF-α ELISA kits (BD Opt EIA) and the percent inhibition of TNF-α release in comparison to the control group was calculated.

TNF-α inhibition results are presented in FIG. 2B.

Conclusion: Oral administration of turmeric at lower doses inhibited LPS induced TNF-α production in Balb/c mice.

### EXAMPLE 3

Effect of a Combination of an Extract of Sphaeranthus indicus (S.I.) and Yashada Bhasma (YB) on LPS Induced TNF-α in Balb/c Mice.

Dose preparation: Required amount of the extract of Sphaeranthus indicus and Yashada bhasma (5 mg/mL+5 mg/mL; 5 mg/mL+10 mg/mL; 5 mg/mL+25 mg/mL) was triturated with Tween 80 until a smooth paste was formed. Carboxymethyl cellulose (0.5%) solution was added gradually with trituration to form a uniform suspension. Yashada Bhasma alone of the concentrations 25 mg/mL, 10 mg/mL and 5 mg/mL was prepared as described in Example 1.

### Conditions for Storage of the Sample

The samples of Sphaeranthus indicus and Yashada bhasma were stored at 22° C. to 25° C.

### Dosing

The mice were randomized (n=6) in the following ten groups of treatment:
i) Group 1: Control group: Mice were administered with vehicle (10 mL/kg, p.o.)
ii) Group 2: Mice administered once daily with 1 mg/kg of lipopolysaccaride i.p.
iii) Group 3: Mice administered once daily with 1.5 mg/kg of Rolipram p.o.
iv) Group 4: Mice administered once daily with 50 mg/kg of extract of Sphaeranthus indicus p.o.
v) Group 5: Mice administered once daily with 50 mg/kg of Yashada Bhasma p.o.
vi) Group 6: Mice administered once daily with 100 mg/kg of Yashada Bhasma p.o.
vii) Group 7: Mice administered once daily with 250 mg/kg of Yashada Bhasma p.o.
viii) Group 8: Mice administered once daily with 50 mg/kg of extract of Sphaeranthus indicus and 50 mg/kg of Yashada Bhasma p.o.
ix) Group 9: Mice administered once daily with 50 mg/kg of extract of Sphaeranthus indicus and 100 mg/kg of Yashada Bhasma p.o.
x) Group 10: Mice administered once daily with 50 mg/kg of extract of Sphaeranthus indicus and 250 mg/kg of Yashada Bhasma p.o.

### Treatment

Extract of Sphaeranthus indicus (10 mL/kg), Yashada bhasma (10 mL/kg), and combination of Yashada bhasma and extract of Sphaeranthus indicus (10 mL/kg) was administered to Balb/c mice of Group 4, Groups 5 to 7, and Groups 8 to 10 respectively.

The positive control group of animals received Rolipram at 1.5 mg/kg. An hour later, lipopolysaccharide (LPS) dissolved in sterile pyrogen-free saline was administered intraperitoneally at a dose of 1 mg/kg to mice of all ten groups. After 1.5 h, post LPS administration, animals were anaesthetized and blood samples were collected by retro orbital method into heparin containing (5 μl) eppendorf tubes. Plasma was separated by centrifugation at 4000 rpm and 4° C. and stored at −80° C. until analysis.

TNF-α levels in the blood samples were assayed using mouse TNF-α ELISA kits (BD Opt EIA) and the percent inhibition of TNF-α release in comparison to the control group was calculated.

TNF-α inhibition results are presented in FIG. 3.

Conclusion: A combination of an extract of Sphaeranthus indicus (S.I.) and Yashada Bhasma (YB) on LPS induced TNF-α in Balb/c mice demonstrated a synergistic response.

### EXAMPLE 4A

Effect of a Combination of Extract of Sphaeranthus indicus (S.I.), Yashada Bhasma (YB) and Turmeric (Tr) on LPS Induced TNF-α in Balb/c Mice

Dose preparation: Required amount of the extract of Sphaeranthus indicus, Yashada bhasma and turmeric (5 mg/mL+5 mg/mL+5 mg/mL, 5 mg/mL+2.5 mg/mL+5 mg/mL, 5 mg/mL+2.5 mg/mL+2.5 mg/mL, 5 mg/mL+2.5 mg/mL+1.25 mg/mL, 5 mg/mL+2.5 mg/mL+0.625 mg/mL, 5 mg/mL+2.5 mg/mL+0.3125 mg/mL) was triturated with Tween 80 until a smooth paste was formed. Carboxymethyl cellulose (0.5%) solution was added gradually with trituration to form a uniform suspension.

### Conditions for Storage of the Sample

The samples of Sphaeranthus indicus, Yashada bhasma and turmeric were stored at 22° C. to 25° C.

### Dosing

The mice were randomized (n=6) in the following nine groups of treatment:
i) Group 1: Control group: Mice were administered with vehicle (10 mL/kg, p.o.)
ii) Group 2: Mice administered once daily with 1 mg/kg of lipopolysaccaride i.p.
iii) Group 3: Mice administered once daily with 1.5 mg/kg of Rolipram p.o.
iv) Group 4: Mice administered once daily with 50 mg/kg of extract of Sphaeranthus indicus, 50 mg/kg of Yashada Bhasma and 50 mg/kg of turmeric p.o.
v) Group 5: Mice administered once daily with 50 mg/kg of extract of Sphaeranthus indicus, 25 mg/kg of Yashada Bhasma and 50 mg/kg of turmeric p.o.
vi) Group 6: Mice administered once daily with 50 mg/kg of extract of Sphaeranthus indicus, 25 mg/kg of Yashada Bhasma and 25 mg/kg of turmeric p.o.
vii) Group 7: Mice administered once daily with 50 mg/kg of extract of Sphaeranthus indicus, 25 mg/kg of Yashada Bhasma and 12.5 mg/kg of turmeric p.o.
viii) Group 8: Mice administered once daily with 50 mg/kg of extract of Sphaeranthus indicus, 25 mg/kg of Yashada Bhasma and 6.25 mg/kg of turmeric p.o.
ix) Group 9: Mice administered once daily with 50 mg/kg of extract of Sphaeranthus indicus, 25 mg/kg of Yashada Bhasma and 3.125 mg/kg of turmeric p.o.

### Treatment

A herbal composition comprising an extract of Sphaeranthus indicus, Yashada bhasma and turmeric (10 mL/kg), was administered to Balb/c mice of Groups 4 to 7. The positive control group (Group 3) of animals received Rolipram at 1.5 mg/kg. An hour later, lipopolysaccharide (LPS) dissolved in sterile pyrogen-free saline was administered intraperitoneally at a dose of 1 mg/kg to mice of all nine groups. After 1.5 h, post LPS administration, animals were anaesthetized and blood samples were collected by retro orbital method into heparin containing (5 μl) eppendorf tubes. Plasma was separated by centrifugation at 4000 rpm and 4° C. and stored at −80° C. until analysis.

TNF-α levels in the blood samples were assayed using mouse TNF-α ELISA kits (BD Opt EIA) and the percent inhibition of TNF-α release in comparison to the control group was calculated.

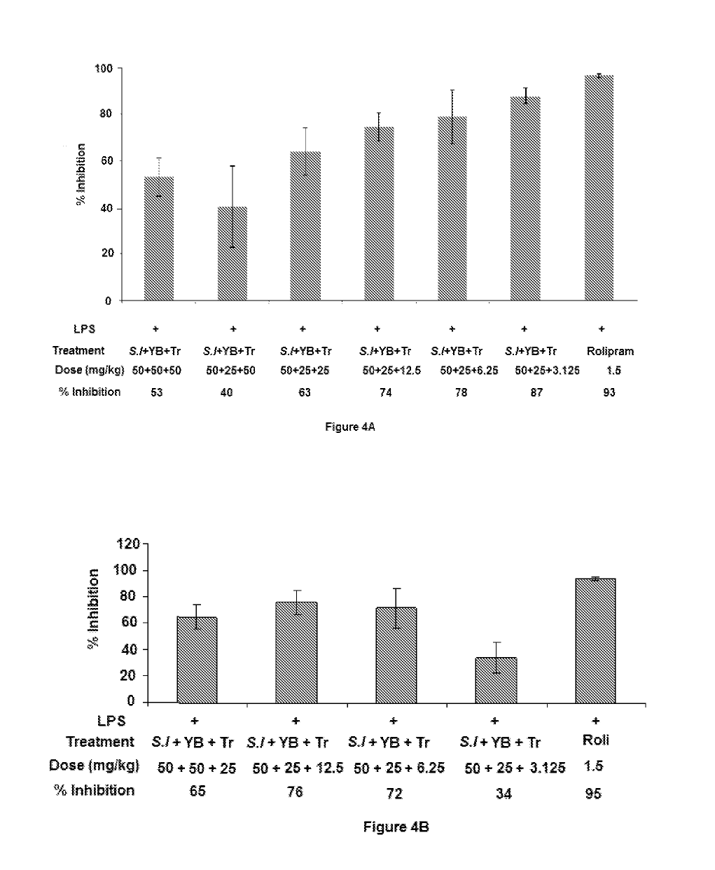

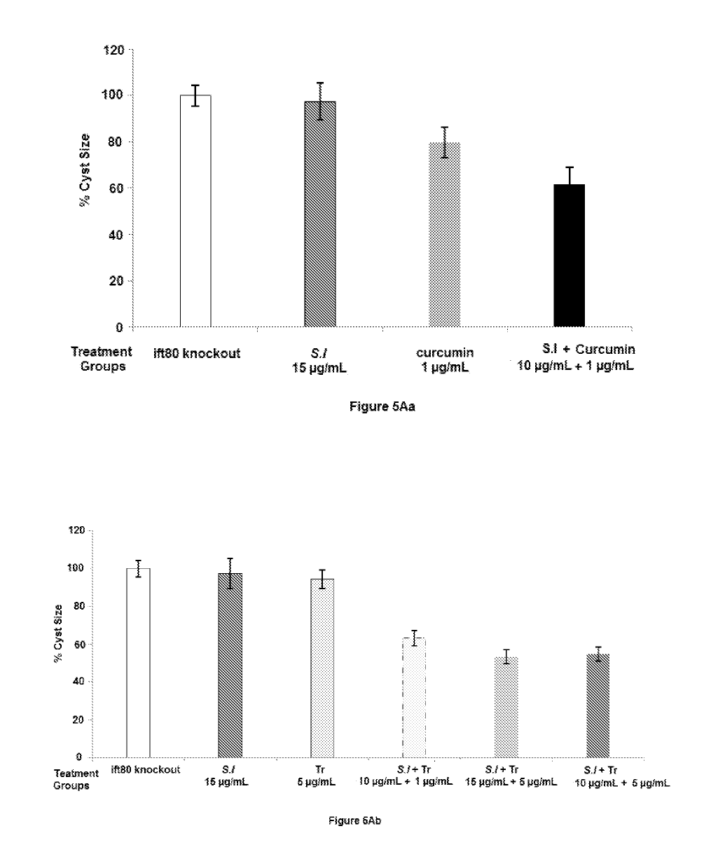

TNF-α inhibition results are presented in FIG. 4A.

Conclusion: A herbal composition comprising Sphaeranthus indicus (S.I.), Yashada bhasma (YB) and turmeric, demonstrated a synergistic response in the reduction of LPS induced TNF-α production.

### EXAMPLE 4B

Effect of a Combination of Extract of Sphaeranthus indicus (S.I.), Yashada Bhasma and Turmeric (containing 25% Curcumin) on LPS Induced TNF-α in Balb/c Mice.

Dose preparation: Required amount of the extract of Sphaeranthus indicus, Yashada bhasma and turmeric (5 mg/mL+5 mg/mL+2.5 mg/mL; 5 mg/mL+2.5 mg/mL+1.25 mg/mL; 5 mg/mL+2.5 mg/mL+0.625 mg/mL, 5 mg/mL+2.5 mg/mL+0.3125 mg/mL) was triturated with Tween 80 until a smooth paste was formed. Carboxymethyl cellulose (0.5%) solution was added gradually with trituration to form a uniform suspension.

### Conditions for Storage of the Sample

Sphaeranthus indicus (S.I.), Yashada Bhasma and turmeric was stored at 22° C. to 25° C.

### Dosing

The mice were randomized (n=6) in the following seven groups of treatment:
i) Group 1: Control group: Mice were administered with vehicle (10 mL/kg, p.o.)
ii) Group 2: Mice administered once daily with 1 mg/kg of lipopolysaccaride i.p.
iii) Group 3: Mice administered once daily with 1.5 mg/kg of Rolipram p.o.
iv) Group 4: Mice administered once daily with 50 mg/kg of extract of Sphaeranthus indicus, 50 mg/kg of Yashada Bhasma and 25 mg/kg of turmeric p.o.
v) Group 5: Mice administered once daily with 50 mg/kg of extract of Sphaeranthus indicus, 25 mg/kg of Yashada Bhasma and 12.5 mg/kg of turmeric p.o.
vi) Group 6: Mice administered once daily with 50 mg/kg of extract of Sphaeranthus indicus, 25 mg/kg of Yashada Bhasma and 6.25 mg/kg of turmeric p.o.
vii) Group 7: Mice administered once daily with 50 mg/kg of extract of Sphaeranthus indicus, 25 mg/kg of Yashada Bhasma and 3.125 mg/kg of turmeric p.o.

### Treatment

Extract of Sphaeranthus indicus (10 mL/kg), Yashada bhasma (10 mL/kg), and combination of Yashada bhasma and extract of Sphaeranthus indicus (10 mL/kg) was administered to Balb/c mice of Group 4, Groups 5 to 7, and Groups 8 to 10 respectively.

The positive control group of animals received Rolipram at 1.5 mg/kg. An hour later, LPS dissolved in sterile pyrogen-free saline was administered intraperitoneally at a dose of 1 mg/kg to mice of all twelve groups. After 1.5 h, post LPS administration, animals were anaesthetized and blood samples were collected by retro orbital method into heparin containing (5 μl) eppendorf tubes. Plasma was separated by centrifugation at 4000 rpm and 4° C. and stored at −80° C. until analysis.

TNF-α levels in the blood samples were assayed using mouse TNF-α ELISA kits (BD Opt EIA) and the percent inhibition of TNF-α release in comparison to the control group was calculated.

TNF-α inhibition results are presented in FIG. 4B.

Conclusion: A herbal composition comprising Sphaeranthus indicus (S.I.), Yashada bhasma (YB) and turmeric (containing 25% curcumin), demonstrated a synergistic response in the reduction of LPS induced TNF-α production.

### EXAMPLE 5

Effect of a Combination of Extract of Sphaeranthus indicus (S.I), Yashada Bhasma, Curcumin and Turmeric on Kidney Cyst Regression

All experiments were carried out with the approval of Institutional Animal Ethics Committee (29/1999/CPCSEA) in Piramal Enterprises Limited, Goregaon, Mumbai, India.

In vivo efficacy of the herbal composition of the present invention on kidney cyst regression was studied on zebrafish morphants. Zebrafish embryos were obtained by wild-type pair matings (Animal House facility of Piramal Enterprises Limited, Mumbai).

The term Zebrafish morphants as used in the following experiments refer to the zebrafish larvae which have developed from zebrafish embryos injected with 1 mM ift80 or 0.25 mM PKD2 or 0.5 μM nek8 translation blocking antisense morpholino oligonucleotides.

### Treatment Groups:

The zebrafish embryos were randomized (n=6) in the following thirteen groups of treatment:
i) Group 1: Zebrafish embryos were administered with 0.1% DMSO
ii) Group 2: Zebrafish embryos were administered with control morpholino oligonucleotides
iii) Group 3: Zebrafish morphants were administered with an extract of Sphaeranthus indicus (15 μg/mL)
iv) Group 4: Zebrafish morphants were administered with curcumin powder (1 μg/mL)
v) Group 5: Zebrafish morphants were administered with turmeric powder (5 μg/mL)
vi) Group 6: Zebrafish morphants were administered with Yashada bhasma (1 μg/mL)
vii) Group 7: Zebrafish morphants were administered with a combination of an extract of Sphaeranthus indicus (10 μg/mL) and curcumin powder (1 μg/mL)
viii) Group 8: Zebrafish morphants were administered with a combination of an extract of Sphaeranthus indicus (10 μg/mL) and turmeric powder (1 μg/mL)
ix) Group 9: Zebrafish morphants were administered with a combination of an extract of Sphaeranthus indicus (15 μg/mL) and turmeric powder (5 μg/mL)
x) Group 10: Zebrafish morphants were administered with a combination of an extract of Sphaeranthus indicus (10 μg/mL) and turmeric powder (5 μg/mL)
xi) Group 11: Zebrafish morphants were administered with a combination of an extract of Sphaeranthus indicus (10 μg/mL), curcumin powder (1 μg/mL) and Yashada bhasma (1 μg/mL)
xii) Group 12: Zebrafish morphants were administered with a combination of an extract of Sphaeranthus indicus (10 μg/mL), turmeric powder (5 μg/mL) and Yashada bhasma (1 μg/mL)
xiii) Group 13: Zebrafish morphants were administered with a combination of an extract of Sphaeranthus indicus (15 μg/mL), turmeric powder (5 μg/mL) and Yashada bhasma (1 μg/mL)

### Treatment:

I. Therapeutic Treatment Modality in ift80 Knockout Zebrafish Morphants

The zebrafish embryos were injected at the two-cell stage with a solution containing 1 mM ift80 translation blocking antisense morpholino oligonucleotides (Gene-Tools, LLC) in 200 mM KCl and 0.1% phenol red to form ift80 knockout zebrafish morphants.

Control Morpholino Oligonucleotides were Injected to Zebrafish Embryos of Group 2.

DMSO (0.1%) was injected to zebrafish embryos of Group 1, as negative control.

24 hours post-fertilization the zebrafish morphants of Groups 3-13 were exposed to drug treatment as described above. The embryos were maintained in 12-well plate and these plates were incubated at 28° C. in an incubator and were read at 24, 48 and at 96 h post fertilization. Embryos were further maintained until 5 days post fertilization and the kidney morphology was documented by immobilization of the larvae in 2.5% methylcellulose and imaged on a Zeiss Axio Observer.A1 inverted microscope Zeiss ((Zeiss AxioCam HSm)). The size of kidney oedema was measured by drawing a polygon around the swelling and calculating the surface area in microns squared using Zeiss Axiolmager 4.5.

### Treatment:

### II. Therapeutic Treatment Modality in PKD2 Knockout Zebrafish Morphants

The zebrafish embryos were injected at the two-cell stage with a solution containing 0.25 mM PKD2 translation blocking antisense morpholino oligonucleotides (Gene-Tools, LLC) in 200 mM KCl and 0.1% phenol red to form PKD2 knockout zebrafish morphants. Control morpholino oligonucleotides were injected to zebrafish embryos of Group 2. DMSO (0.1%) was injected to zebrafish embryos of Group 1, as negative control. 24 hours post-fertilization the zebrafish morphants of Groups 3 to 6 and 11 to 13 were exposed to drug treatment as described above. The embryos were maintained in 12-well plate and these plates were incubated at 28° C. in an incubator and were read at 24, 48 and at 96 hours post fertilization. Embryos were further maintained until 5 days post fertilization and the kidney morphology was documented by immobilization of the larvae in 2.5% methylcellulose and imaged on a Zeiss Axio Observer.A1 inverted microscope Zeiss ((Zeiss AxioCam HSm)). The size of kidney oedema was measured by drawing a polygon around the swelling and calculating the surface area in microns squared using Zeiss Axiolmager 4.5.

III. Therapeutic Treatment Modality in nek8 Knockout Zebrafish Morphants

The zebrafish embryos were injected at the two-cell stage with a solution containing 0.5 μM nek8 translation blocking antisense morpholino oligonucleotides (Gene-Tools, LLC) in 200 mM KCl and 0.1% phenol red to form nek8 knockout zebrafish morphants. Control morpholino oligonucleotides were injected to zebrafish embryos of Group 2. DMSO (0.1%) was injected to zebrafish embryos of Group 1, as negative control. 24 hours post-fertilization the zebrafish morphants of Groups 3 to 6 and 11 to 13 were exposed to drug treatment as described above. The embryos were maintained in 12-well plate and these plates were incubated at 28° C. in an incubator and were read at 24, 48 and at 96 h post fertilization. Embryos were further maintained until 5 days post fertilization and the kidney morphology was documented by immobilization of the larvae in 2.5% methylcellulose and imaged on a Zeiss Axio Observer.A1 inverted microscope Zeiss ((Zeiss AxioCam HSm)). The size of kidney oedema was measured by drawing a polygon around the swelling and calculating the surface area in microns squared using Zeiss Axiolmager 4.5.

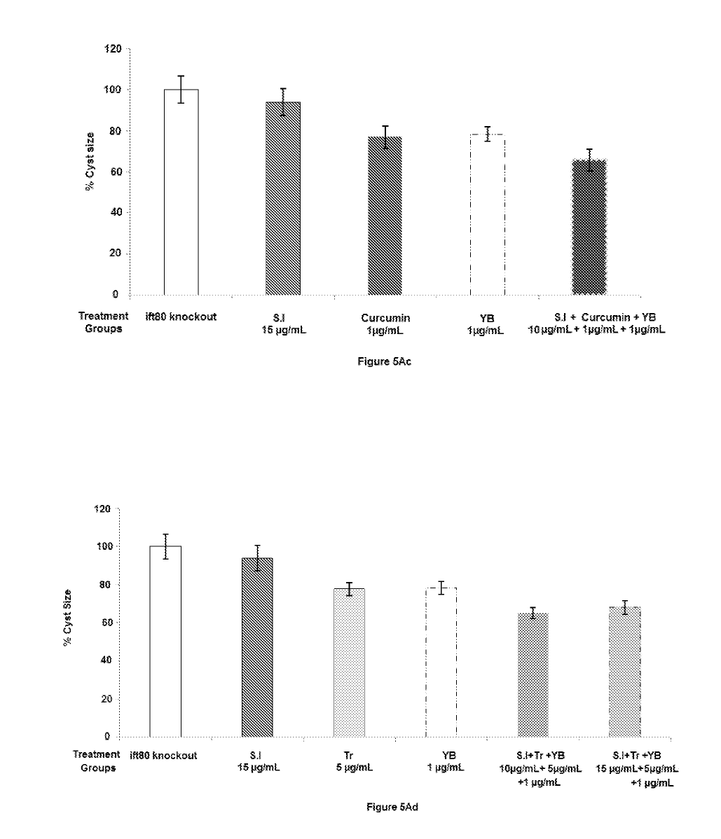

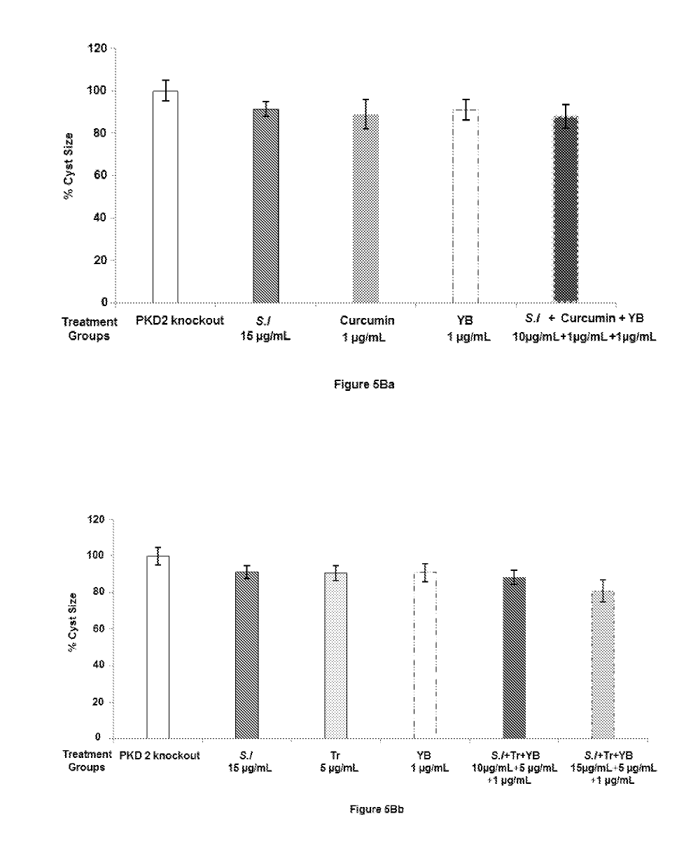

Results: FIG. 5Aa, FIG. 5Ab, FIG. 5Ac, FIG. 5Ad, FIG. 5Ba, FIG. 5Bb, FIG. 5Ca and FIG. 5Cb show the effect of combination of extract of Sphaeranthus indicus (S.I), Yashada Bhasma, curcumin and turmeric on kidney cyst regression.

Conclusion: A herbal composition comprising Sphaeranthus indicus (S.I.), Yashada bhasma (YB) and turmeric (containing 25% curcumin), demonstrated a synergistic response on kidney cyst regression.

### EXAMPLE 6

Effect of a Combination of Extract of Sphaeranthus indicus (S.I), Yashada Bhasma and Turmeric on Chronic Ear Inflammation

### Animals Used:

Balb/c male mice, 8 to 12 weeks old, weighing 22 to 28 g (Animal House facility of Piramal Enterprises Limited, Mumbai). Animals were housed in individually ventilated polypropylene cages under specified pathogen-free conditions maintained at 18 to 24° C. and 50 to 70% humidity, with a 12-hour light/12-hour dark cycle. The mice were acclimatized for a period of one week before experimentation. Animals were handled in a laminar flow hood. Mice had access to food and purified water ad libitum.

### Conditions for Storage of the Compounds

Sphaeranthus indicus (S.I.), Yashada Bhasma, turmeric and the standard compound were stored at room temperature (22° C. to 25° C.).

### Drug Administration

The mice were randomized (n=10) in the following five groups of treatment:
i) Group 1: Mice were topically applied with placebo cream (25 mg)
ii) Group 2: Control group: Mice were topically applied with betamethasone valerate cream BP (25 mg)
iii) Group 3: Mice were topically applied with Sphaeranthus indicus (S.I.) cream (5%) alone (25 mg)
iv) Group 4: Mice were topically applied with composition A (Sphaeranthus indicus (5%), Yashada bhasma (2.5%) and turmeric (0.3%)), (25 mg)
v) Group 5: Mice were topically applied with composition B (Sphaeranthus indicus (5%), Yashada bhasma (2.5%) and turmeric (0.6%)), (25 mg).

### Preparation of Inflammation Inducing Agent

4 μg of phorbol 12-myristate 13-acetate (PMA) (Sigma Aldrich Inc., USA) dissolved in 20 μL of acetone to form PMA solution. The solution was stored at −20° C.

### Treatment

Male Balb/c mice (8-12 weeks of age, body weight between 22-28 g) were acclimatized to laboratory conditions for one week prior to the initiation of the experiment. On day 0, ear thickness of both left and right ear of all the mice were measured using caliper, POCO 2T. After recording ear thickness, PMA solution was applied to the inner and outer surface of right ears of all the animals of Groups 1 to 5. Thirty minutes after the application of PMA, the inner and outer surface of the ear (12.5 mg to each side) was applied with placebo cream, Sphaeranthus indicus cream (5%), betamethasone valerate cream BP, Composition A and Composition B to the animals of Groups 1, 2, 3, 4 and 5 respectively. The procedure was repeated through day 1 to 10. Animals were humanely euthanized on day 11 after recording body weight and ear thickness.

Percent increase in the thickness of ear was calculated by comparing ear thickness on respective days with the basal ear thickness on day 0. A graph was plotted with days of the study on x-axis and percentage increase in ear thickness on y-axis. Area under curve (AUC) of the graph of different treatments was compared with AUC of placebo group.

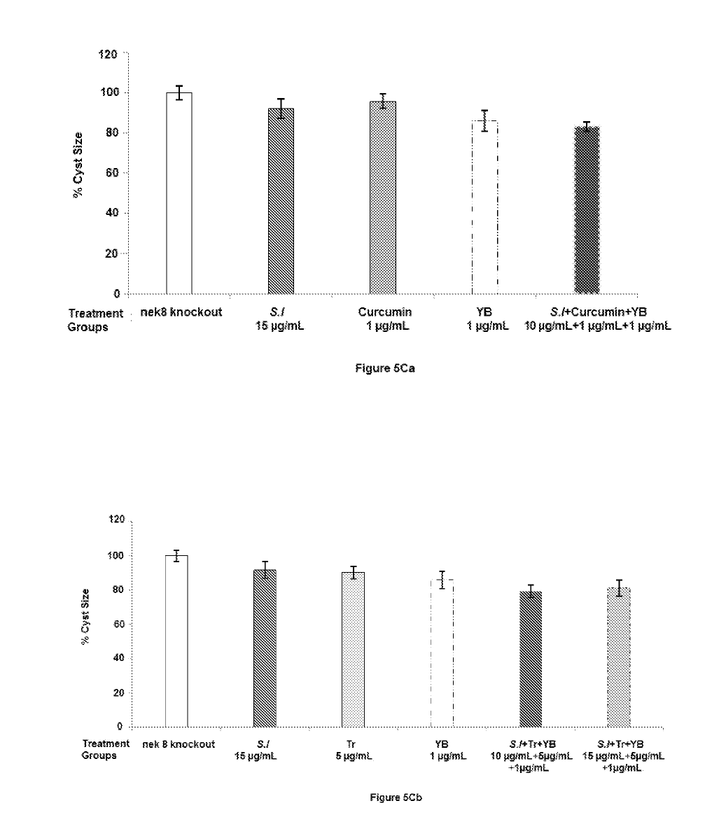

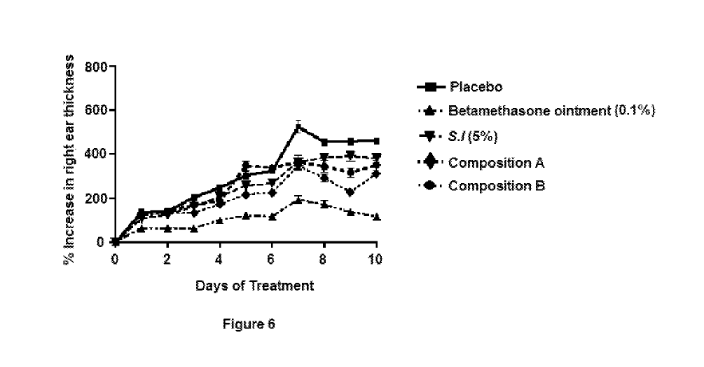

FIG. 6 shows the effect of Composition A and Composition B on PMA induced ear inflammation.

Table 1 shows the effect of different treatments on PMA induced ear inflammation.

| | AUC of different treatments | | | | |
|---|---|---|---|---|---|
| | | | Sphaeranthus | | |
| Animal | Placebo | Betamethsone | indicus cream | Composition | Composition |
| Groups | cream | ointment | (5%) | (A) | (B) |
| 1 | 3050.0 | 1333.0 | 2500.0 | 1783.0 | 2783.0 |
| 2 | 2850.0 | 1300.0 | 2650.0 | 1767.0 | 2183.0 |
| 3 | 3200.0 | 800.0 | 2383.0 | 1967.0 | 2400.0 |
| 4 | 3100.0 | 783.3 | 2717.0 | 1767.0 | 2717.0 |
| 5 | 3533.0 | 1033.0 | 2633.0 | 1933.0 | 2267.0 |
| 6 | 3017.0 | 950.0 | 2367.0 | 2150.0 | 2117.0 |
| 7 | 3267.0 | 1183.0 | 2183.0 | 2050.0 | 2133.0 |
| 8 | 2750.0 | 866.7 | 2700.0 | 2067.0 | 2600.0 |
| 9 | 2800.0 | 1317.0 | 2350.0 | 2267.0 | 2833.0 |
| 10 | 2600.0 | 1283.0 | 2017.0 | 2400.0 | 3050.0 |
| Average | 3017.0 | 1085.0 | 2450.0 | 2015.0 | 2508.0 |
| SEM | 87.58 | 70.80 | 73.78 | 68.29 | 105.2 |
| P value | — | p < 0.001 | p < 0.001 | p < 0.001 | p < 0.001 |
| w.r.t. placebo | | | | | |
| cream | | | | | |

Conclusion: Composition A and Composition B demonstrate a significant amelioration of ear inflammation when compared to placebo cream.

Thus composition A and composition B are potential alternatives for the treatment of psoriasis.

Reference: Kimiko Nakajima et al. (The Journal of Immunology, 2011, 187(11), 6157-8) have successfully demonstrated the use of phorbol 12-myristate 13-acetate (PMA)-induced ear model to screen compounds for psoriasis.

1. A herbal composition comprising at least two active ingredients selected from an extract of flowering and/or fruiting heads of Sphaeranthus indicus, Yashada bhasma and curcumin or curcumin containing substance.

2. The herbal composition according to claim 1, comprising the active ingredients selected from an extract of flowering and/or fruiting heads of Sphaeranthus indicus, Yashada bhasma and curcumin or curcumin containing substance.

3. The herbal composition according to claim 1, comprising active ingredients selected from Yashada bhasma and an extract of flowering and fruiting heads of Sphaeranthus indicus.

4. The herbal composition according to claim 1, comprising active ingredients selected from Yashada bhasma and curcumin or curcumin containing substance.

5. The herbal composition according to claim 1, comprising active ingredients selected from an extract of flowering and fruiting heads of Sphaeranthus indicus and curcumin or curcumin containing substance.

6. The herbal composition according to claim 1, wherein the composition further comprises a pharmaceutically acceptable carrier or excipient.

7. The herbal composition according to claim 2, comprises of an extract of flowering and fruiting heads of Sphaeranthus indicus, Yashada bhasma and curcumin containing substance in the ratio 1:1:1 to 16:8:5.

8. The herbal composition according to claim 3, comprises of an extract of flowering and fruiting heads of Sphaeranthus indicus and Yashada bhasma in the ratio 1:1 to 1:5.

9. The herbal composition according to claim 5, comprises of an extract of flowering and fruiting heads of Sphaeranthus indicus and curcumin or curcumin containing substance in the ratio 1:1 to 10:5.

10. The herbal composition according to claim 2, comprising
(a) from about 30% to 70% by weight of an extract of flowering and fruiting heads of Sphaeranthus indicus
(b) from about 20% to about 40% of Yashada bhasma
(c) from about 3% to about 40% of curcumin or curcumin containing substance.

11. A herbal composition according to claim 3, comprising
(a) from about 10% to 50% by weight of an extract of flowering and fruiting heads of Sphaeranthus indicus and
(b) from about 50% to about 85% of Yashada bhasma.

12. The herbal composition according to claim 1, wherein the curcumin containing substance comprises of about 20% to about 30% of curcumin.

13. The herbal composition according to claim 12, wherein the curcumin containing substance is turmeric.

14. A method for the prevention or treatment of a disease mediated by TNF-α (tumor necrosis factor-α) comprising administering to a subject in need thereof a therapeutically effective amount of the herbal composition according to claim 1.

15. The method according to claim 14, wherein the disease mediated by TNF-α is an inflammatory disease or a disease having an inflammatory component or renal cystic disease.

16. The method according to claim 15, wherein the inflammatory disease or a disease having an inflammatory component is selected from the group consisting of coronary heart disease, atherosclerosis, hyperlipidemia ischemia-reperfusion injury, stroke, cachexia, hypertension, vasculitis, Wegener's granulomatosis multiple sclerosis, common variable immunodeficiency (CVID), skin delayed type hypersensitivity disorders, ankylosing spondylitis, rheumatoid arthritis, juvenile rheumatoid arthritis, psoriatic arthritis, osteoarthritis, refractory rheumatoid arthritis, chronic non-rheumatoid arthritis, chronic graft-versus-host disease, psoriasis, conjunctivitis, Crohn's disease, ulcerative colitis, Behcet's disease, inflammatory bowel disease, osteoporosis/bone resorption, chronic obstructive pulmonary disease and asthma.

17. The method according to claim 16, wherein inflammatory disease or a disease having an inflammatory component is selected from the group consisting of rheumatoid arthritis, juvenile rheumatoid arthritis, psoriatic arthritis, osteoarthritis, refractory rheumatoid arthritis, chronic non-rheumatoid arthritis, psoriasis, ulcerative colitis, inflammatory bowel disease, osteoporosis/bone resorption, Crohn's disease, atherosclerosis and ankylosing spondylitis.

18. The method according to claim 15, wherein the renal cystic disease is polycystic kidney disease.

19. The method according to claim 17, wherein the polycystic kidney disease is an autosomal dominant polycystic kidney disease or autosomal recessive polycystic kidney disease.
